(19) United States
(12) Patent Application Publication
(10) Pub. No.: US 2024/0183776 A1
(43) Pub. Date: Jun. 6, 2024

(54) Analysis System for the Determination of Ions in an Ion-Containing Liquid Medium, and an Analysis Process Performed with the Analysis System

(71) Applicants: Leonid Guggenberger, Ravensburg (DE); Lukas-David Hamm, Rheinau (DE)

(72) Inventor: Lukas-David Hamm, Rheinau (DE)

(21) Appl. No.: 18/551,275

(22) PCT Filed: Mar. 18, 2022
(86) PCT No.: PCT/EP2022/057206

§ 371 Date: Sep. 19, 2023

(30) Foreign Application Priority Data
Mar. 19, 2021 (DE) 10 2021 001 442.1

(51) Int. Cl. G01N 21/31; G01N 1/14; G01N 1/40

(52) U.S. Cl. CPC G01N 21/31; G01N 1/14; G01N 1/4005

(57) ABSTRACT

The present invention relates to an analysis system for the qualitative and/or quantitative determination of ions in an ion-containing liquid medium. The present invention also relates to an analysis process performed with said analysis system. With the proposed analysis system and analysis process, ions in a wide variety of ion-containing liquid media can be qualitatively and/or quantitatively determined in an easy and inexpensive way.

### CROSS REFERENCE TO RELATED APPLICATIONS

This application is a U.S. National Application of PCT/EP2022/057206 filed Mar. 18, 2022, which published as PCT Publication WO2022/195093, which is incorporated by reference herein in its entirety.

### FIELD OF THE INVENTION

The present invention relates to an analysis system for the qualitative and/or quantitative determination of ions in an ion-containing liquid medium. The present invention also relates to an analysis process performed with said analysis system.

### BACKGROUND OF THE INVENTION

Currently, ion-selective probes are often used to determine ionic parameters in aqueous solution and to be able to monitor the parameters automatically by means of probes. The probes are each able to determine one ionic parameter from its redox potential in the solution. In this context, ion selectivity is achieved through the use of ion-selective membranes.

However, in practice these probes are associated with a number of serious drawbacks. For example, the electrodes are prone to cross sensitivities. This means that the electrodes typically do not have full selectivity, and consequently measure interfering ions as well, which distorts the measurement results. Another problem is the extreme sensitivity to environmental influences of the membranes used. Ion-selective probes are also expensive, which places them out of reach for certain (e.g., private) users. Finally, probes of such kind must be recalibrated regularly, resulting in substantial servicing work, particularly if they are in continuous use.

The increasing pollution of waters is a global problem which is also very prevalent in technologically advanced First World countries. One cause of this problem is the rise of industrialisation and commercialisation of the agricultural sector. Accordingly, in order to meet the worldwide need for food, agriculture must produce more and more food in less and less space. The growing demand for biomass in the pursuit of climate-neutral energy generation exacerbates the situation. In order to be able to meet this need, agriculture must then resort to man-made fertilisers, which keep farmland fertile even when it is exposed to extremely high demands. The use of fertilisers also serves to enhance the growth of crops and thus increase overall yield. However, the fertilisers are not fully absorbed by the crops, and the excess fertiliser is carried into the surrounding bodies of water and the groundwater by the rain, which can have serious consequences for the local ecosystems.

Thus, an overabundance of plant nutrients results in increased algae formation. Dead algae sink to the bottom of the water, where they are typically used by aerobic decomposition processes. In the event of excessive algal growth, more algae also die, and more oxygen is needed to decompose this biomass. This in turn can be fatal for other organisms living in the water, such as fish, and may cause a mass fish die-off, for example. Over time, when there is no more oxygen in the water, biomass decomposition takes place anaerobically, that is to say without oxygen, which also causes the formation of harmful marsh gases and is referred to colloquially as an aquatic “dead zone”.

However, overfertilisation is not only harmful to the ecosystems it affects, it can also pose a direct threat to humans. For example, the nitrogen contained in the fertilisers can get into the groundwater in the form of nitrate, and from there into our drinking water. Upon conversion into nitrite in our bodies, the nitrate can again cause health problems.

In order to be able to forestall overfertilisation, bodies of water and watercourses must be monitored constantly. This would enable fertilisation to be optimised, and it would also be possible to respond rapidly to excessively high fertiliser concentrations. However, the processes currently in use do not lend themselves to automatic monitoring of large expanses of water. It would also be of interest to monitor the fertiliser concentrations in real time and regulate the addition of fertilisers fully automatically. Unfortunately, current automatic measuring systems and processes are too expensive, for such an application; they also require intensive maintenance and are prone to malfunction.

Furthermore, the monitoring of ion contents and identification of certain types of ions in liquid system is also of interest in other application fields, for example in water treatment plants, sewage plants, industrial plants etc. Another pertinent application area for the monitoring of ion contents concerns fishkeeping, i.e. the qualitative and/or qualitative determination of ions in ion-containing liquid media that are used in aquaria. Indeed, it is in aquaria that plant types and animal types (fish, small animals etc.) are kept which are sensitive to certain environmental conditions (e.g. even the ion content in the water of the aquarium). A reliable, easily manageable method for the qualitative and quantitative monitoring of the ion types contained in them is therefore of great interest.

### SUMMARY OF THE INVENTION

In view of the above, the problem addressed by the invention is the development of an analysis system and an analysis process for the determination of ions in an ion-containing liquid medium with which the drawbacks of the analysis systems and processes used in the prior art are avoided. Accordingly, it should be possible to manufacture and operator the analysis system and analysis process suggested according to the invention than the systems known from the prior art. The analysis system and analysis process should also be less sensitive to interference ions and require less maintenance effort. The analysis system and analysis process should also be easy to operate.

As stated earlier, the present invention relates to an analysis system for the determination of ions in an ion-containing liquid medium.

In this context, an ion-containing liquid medium may be understood to be matter in a liquid aggregate state that can contain ions. The liquid ion-containing medium may be for example an ion-containing liquid, e.g., a liquid solution. In such as case, an ion-containing liquid solution may be understood to be a homogeneous mixture that contains ions of at least one ion type dissolved in a solvent. The solvent in such case may be for example water, and the solution may accordingly be an aqueous solution. The ion-containing liquid medium to be analysed may be for example a sample taken from body of standing or flowing water, such as a lake or river. The ion-containing liquid medium to be analysed may equally originate from a standing or flowing reservoir of a sewage plant, a water treatment plant, an aquarium or an industrial plant.

In this context, ions may generally be understood to be electrically charged particles that may form from uncharged atoms or molecules by losing or receiving electrons. Cations may be understood to be positively charged ions which form from uncharged atoms or molecules due to the loss of electrons. On the other hand, anions may be understood to be negatively charged ions which are formed from uncharged atoms or molecules by accepting electrons. Anions and cations may be monovalent or polyvalent.

The ions are generally determined in qualitative and/or quantitative terms. The determination of the ions may thus be performed both qualitatively and quantitatively. Alternatively, the ions may be determined only quantitatively, or only qualitatively. In the context of the present disclosure, a “quantitative” determination is understood to be a determination based on quantity, for example a quantity (also amount of a substance) per volume, an absolute quantity (also amount of a substance, mass or the like), a relative quantity relative to a comparison measurement, or the like. On the other hand, a “qualitative” determination is understood to be a classification according to kind/type of an ion species, for example to address the question of whether a certain ion type is present or not.

The suggested analysis system for determination of the ions present in the ion-containing liquid medium comprises a measuring cell and a measuring arrangement.

In this context, the measuring cell of the analysis system comprises a first measuring chamber, a second measuring chamber, and a sample chamber arranged between the first measuring chamber and second measuring chamber, which is designed to hold the ion-containing liquid medium. A first electrode is arranged in the first measuring chamber, and a second electrode is arranged in the second measuring chamber.

Here, an electrode may generally be understood to be any electrical conductor in solid form which is able to conduct an electrical charge carrier. In this context, “conducting” may be understood to refer to the transport of electrical charge carriers, such as electrons or ions. Electrons may be understood to be electrically charged elementary particles that have a negative charge. The electrode may be made from a conductive material, graphite for example, or a metal such as titanium or platinum. At an electrode, electrical charge carriers can be transferred from the solid from which the electrode is made to the medium surrounding the electrode, or electrical charge carriers can be taken up from said medium.

In this situation, a voltage may be applied between the first and the second electrode in such a way that the first electrode is positively polarised (charged) and the second electrode is negatively polarised, so that the anions are transported towards the first electrode and the cations are transported towards the second electrode. The term “transporting” of ions may be understood to mean for example a transport of ions in a liquid medium.

In principle, any electrical voltage source designed to generate an electrical voltage between electrodes may be used to generate an electrical voltage between the first and the second electrode. An electrical voltage source may be understood to be an active two-terminal network that generates an electrical voltage between its terminal points. In this context, a two-terminal network may be understood to be an electrical component or an electrical circuit with two connections (terminals, poles).

The electrical voltage source may be a battery or a generator for example. In order to generate a voltage between the first and the second electrode, the electrical voltage source may be connected to the first electrode via a first electrical conductor and to the second electrode via a second electrical conductor. Depending on the electrical polarity between the first and the second electrode, a condition of either electron deficiency or electron surplus may exist at the first and second electrodes respectively. The electrode may be positively or negatively polarised accordingly.

In addition, an anion-selective membrane designed to enable anions to pass out of the sample chamber into the first measuring chamber is arranged between the first measuring chamber and the sample chamber.

In addition, a cation-selective membrane designed to enable cations to pass out of the sample chamber into the second measuring chamber is arranged between the second measuring chamber and the sample chamber.

The first measuring chamber is filled with a first liquid measuring chamber medium. The first liquid measuring chamber medium may contain a first detection substance in order to form anion detection substance associates when anions pass through into the first measuring chamber. The second measuring chamber is also filled with a second liquid measuring chamber medium. The second liquid measuring chamber medium may contain a second detection substance, in order to form cation detection substance associates when cations pass through into the second measuring chamber.

In the context of the present invention, a liquid measuring chamber medium may be understood to be any medium present in a liquid aggregate state, with which a measuring chamber can be filled. In such a case, the first liquid measuring chamber medium may be understood to be such a liquid measuring chamber medium, with which the first measuring chamber is filled. In turn, the second liquid measuring chamber medium may be understood to be such a liquid measuring chamber medium, with which the second measuring chamber is filled. In this context, the first liquid measuring chamber medium and das second liquid measuring chamber medium may correspond to the ion-containing liquid medium. A “filling” may be understood to mean complete filling of the respective measuring chamber with the respective liquid measuring chamber medium, or equally an only partial filling (in this case, the respective measuring chamber is not completely filled with liquid measuring chamber medium relative to its volume).

The analysis system further comprises a measuring arrangement. The measuring arrangement is designed to measure the anions and/or anion detection substance associates present in the first measuring chamber, and/or the cations and/or cation detection substance associates present in the second measuring chamber photometrically as a photometric measurement. This may be understood on the basis of the following three alternatives.

In a first alternative, the anions and/or anion detection substance associates present in the first measuring chamber may be measured photometrically with the measuring arrangement by way of a photometric measurement. Thus, anions and anion detection substance associates may be measured photometrically. Alternatively, only anions, or only anion detection substance associates may be measured photometrically. The anions are then determined qualitatively and/or quantitatively on the basis of the respective measurement(s). This may be understood to mean that the determination of the anions is carried out qualitatively and quantitatively. Alternatively, the anions may be determined only quantitatively, or only qualitatively.

Alternatively, the cations and/or cation detection substance associates present in the second measuring chamber may be measured photometrically with the measuring arrangement by way of a photometric measurement. This may be understood to mean that cations and cation detection substance associates are measured photometrically. Alternatively, only cations or only cation detection substance associates may be measured photometrically. The cations are then determined qualitatively and/or quantitatively on the basis of the respective photometric measurement(s). This may be understood to mean that the determination of the cations is carried out qualitatively and quantitatively. Alternatively, the cations may be determined only quantitatively, or only qualitatively.

In addition, both the anions and/or anion detection substance associates present in the first measuring chamber, and the cations and/or cation detection substance associates present in the second measuring chamber may be measured with the measuring arrangement. The anions and the cations are then each determined qualitatively and/or quantitatively on the basis of the respective photometric measurement(s). This may be understood to mean that the determination of the anions and cations is carried out both qualitatively and quantitatively. Alternatively, anions and cations may be determined only quantitatively, or only qualitatively.

The analysis system is advantageously universally usable and fully adjustable. This can be understood to mean that the analysis system may be designed such that anions and/or cations can be measured quantitatively and/or qualitatively in the first and/or second measuring chamber. The analysis system is advantageously also of relatively simple construction and easy to operate, and therefore lends itself to use by an end consumer (layperson).

Because of its simple construction, the analysis system can also be manufactured and maintained inexpensively.

The subordinate claims relate to advantageous variants and further developments of the present invention. The features described in the subordinate claims may be implemented in any combination to develop the analysis system according to the invention further, if such development is technically feasible. This still applies if combinations of such kind are not illustrated explicitly with corresponding references in the claims. In particular, this also remains applicable beyond the category limits of the claims, to the effect that those features and/or variants described in the context of the analysis system may also be features and/or variants of the analysis process which is also suggested with the invention, and vice versa.

According to a first variant of the invention, a first mixing apparatus may be arranged in the first measuring chamber, and/or a second mixing apparatus may be arranged in the second measuring chamber to thoroughly mix the first or second liquid measuring chamber medium contained in the first or second measuring chamber. This may be understood to mean that, in a first alternative, a first mixing apparatus is arranged in the first measuring chamber to thoroughly mix the first liquid measuring chamber medium contained in the first measuring chamber, and a second mixing apparatus is arranged in the second measuring chamber to thoroughly mix the second liquid measuring chamber medium contained in the first measuring chamber. In a second alternative, there is only a first mixing apparatus arranged in the first measuring chamber. In a third alternative, there is only a second mixing apparatus arranged in the second measuring chamber. In general, a mixing apparatus may be any apparatus that is suitable for thoroughly mixing the respective chamber media. The mixing apparatus may be of a mechanical type (e.g., an agitator, a stirring bar with magnetic stirrer), but equally it may also be based on a throughflow or ultrasonic mixing. Any mixing or agitation apparatuses known from the prior art may be considered a “mixing apparatus” within the meaning of this invention. Good mixing may improve the measurement accuracy.

According to a further variant of the invention, a pH of the first liquid measuring chamber medium contained in the first measuring chamber and/or of the second liquid measuring chamber medium contained in the second measuring chamber may be adjusted. This may be understood to mean that a pH of the first liquid measuring chamber medium contained in the first measuring chamber and of the second liquid measuring chamber medium contained in the second measuring chamber are both adjusted. Alternatively, only a pH of the first liquid measuring chamber medium contained in the first measuring chamber, or only a pH of the second liquid measuring chamber medium contained in the second measuring chamber may be adjusted.

The adjustment of the pHs of the first liquid measuring chamber medium contained in the first measuring chamber and/or of the second liquid measuring chamber medium contained in the second measuring chamber is important because the optionally utilised detection substances each form the desired photometrically detectable ion detection substance associates with the ions that are to be investigated in certain pH conditions. Thus for example, certain detection substances are only able to form said ion detection substance associates with the ions that are to be investigated when the measuring chamber medium has an alkaline pH. On the other hand, other detection substances need a measuring chamber medium with an acidic pH in order to be able to form said ion detection substance associates with the ions that are to be investigated. The pH of a medium may be understood to be a measure of the acidic or basic nature of the medium. At the same time, the pH may represent the additive inverse of the common logarithm of hydrogen ion activity (i.e., the higher the concentration of hydrogen ions in a medium, the lower its pH is) and have a value between 0 and 14. In this context, a pH<7 may correspond to a medium with acid effect, a pH=7 may correspond to a neutral medium, and a pH>7 may correspond to a medium with basic effect.

If the measuring chamber media contain water, the adjustment of the pH of the first and second measuring chamber media may be initiated by water electrolysis taking place at the electrodes. In the course of water electrolysis, water may be converted into hydrogen and oxygen by electrochemical processes, wherein oxonium ions (H3O+) or hydroxide ions (OH−) are formed or consumed depending on the conditions in the medium. For example, if a voltage is applied between two electrodes, water will yield hydrogen, which collects at a negatively charged electrode while oxygen collects at a positively charged electrode.

For example, when a voltage is passed between the first and second electrodes, causing the first electrode to become positively polarised and the second electrode to be negatively polarised, during water electrolysis a direct current will flow from the negatively charged second electrode to the positively charged first electrode. In this context, a direct current flow may be understood as a migration of electrical charge carriers such as electrons through ions dissolved in the water. In this situation, as a result of the electrochemical processes that take place during the water electrolysis, in the second measuring chamber, where the negatively charged second electrode is arranged, water may become basic and electron-rich (hydrogen-saturated), and in the first measuring chamber, where the positively charged first electrode is arranged, water may become acidic and electron-poor (oxygen-saturated).

For example, water electrolysis may be performed in such manner that an alkaline pH is created in the second measuring chamber, where the negatively charged electrode is arranged. The alkaline pH established may then favour the capability of a detection substance to form photometrically detectable ion detection substance associates. For example, the capability of the metal indicator calmagite (which can supply a detection substance) to form photometrically detectable cation-metal indicator complexes may be enhanced by establishing an alkaline pH in the second liquid measuring chamber medium containing calmagite as the detection substance.

In principle, any possible water electrolysis arrangement that is designed to perform a water electrolysis may be used to adjust the pH of the medium in the first and second liquid measuring chambers. In the following text, three alternative versions of a possible water electrolysis arrangement are described. However, it should be noted explicitly at this point that these exemplary descriptions, do not represent a limitation to certain water electrolysis arrangements.

In a first alternative, in order to adjust pH of the first liquid measuring chamber medium contained in the first measuring chamber and of the second liquid measuring chamber medium contained in the second measuring chamber, a charge-permeable membrane may be arranged between the first measuring chamber and the second measuring chamber, to enable a charge to pass between the first measuring chamber and the second measuring chamber. In this context, any membrane that allows the electrical charge carriers such as electrons, oxonium ions (H3O+) or hydroxide ions (OH−) to pass through the charge-permeable membrane may be understood to be a charge-permeable membrane. In principle, all known charge-permeable membranes may be used.

In a second alternative, a first charge-permeable membrane is arranged between the first measuring chamber and a first water electrolysis chamber equipped with a first water electrolysis electrode, to enable a charge to pass between the first measuring chamber and the first water electrolysis chamber. In this case, a water electrolysis cell is created from the first measuring chamber and the first water electrolysis chamber. The first water electrolysis electrode and the first electrode form an essential water electrolysis cell electrode pair. And the first electrode forms a further water electrolysis electrode of this water electrolysis cell as well as the first water electrolysis electrode. In this example, the first water electrolysis chamber and the first measuring cell are preferably filled with water.

Alternatively or additionally, a second charge-permeable membrane may be arranged between the second measuring chamber and a second water electrolysis chamber equipped with a second water electrolysis electrode, to enable a charge to pass between the second measuring chamber and the second water electrolysis chamber. In this case, a water electrolysis cell is created from the second measuring chamber and the second water electrolysis chamber. The second water electrolysis electrode and the second electrode form an essential water electrolysis cell electrode pair. And the second electrode forms a further water electrolysis electrode of this water electrolysis cell as well as the second water electrolysis electrode. In this example, the second water electrolysis chamber and the second measuring cell are preferably filled with water. Regarding the functional principle of water electrolysis, reference is herewith made to the earlier notes.

The effect of adjusting the pH on the basis of the water electrolysis principle is that there is no need for electrochemical pre-amplification (applying a voltage to the first and second electrodes, together with ion migration resulting therefrom and possibly then forming ion detection substance associates) to bring the pH into the range in which the detection substances enable formation of ion detection substance associates and then enable photometric detection thereof. This is particularly advantageous for high concentrations of ions in the liquid, ion-containing medium. In the case of low ion concentrations, however, electrochemical pre-amplification is advantageous in order to increase the respective ion concentrations in the measuring chambers and at the electrodes arranged in the measuring chambers, to enable a reliable photometric measurement to take place.

In a third alternative, the measuring cell may include a pH adjustment chamber for adjusting the pH in the first and second measuring chambers, wherein a first charge-permeable membrane is arranged between the first measuring chamber and the pH adjustment chamber, and wherein a second charge-permeable membrane is arranged between the second measuring chamber and the pH-adjustment chamber. In this context too, a charge-permeable membrane may be understood to be any membrane that allows electrical charge carriers such as electrons, oxonium ions (H3O+) or hydroxide ions (OH−) to pass through the charge-permeable membrane. With this chamber arrangement and by using the first and second electrodes—when a voltage is applied to these—the pH can be adjusted by carrying out a water electrolysis. In this example, the first and second electrodes form a water electrolysis electrode pair necessary for performing the water electrolysis. Regarding the functional principle of water electrolysis reference is made to the earlier notes.

Unlike the water electrolysis, such a construction may also serve to carry out a different pH adjustment process which makes use of the first and second electrodes. In this context, a medium other than water may be present in the pH adjustment chamber. In this example, the first and second charge-permeable membranes may be an anion-selective and a cation-selective membrane.

According to a further variant of the invention, the measuring arrangement may comprise a light source, a measuring space, a first optical waveguide arranged between the light source and the measuring space, a second optical waveguide arranged between the measuring space and a detector, and an evaluation unit.

In this context, the light source may be designed to generate one or more light beam(s). The light source may be designed to generate one or more light beam(s) of a certain wavelength, for example. For example, the light source may be a LED lamp, a deuterium lamp (UV, ultraviolet light), a xenon flash lamp (UV, VIS) or a tungsten halogen lamp (VIS, visible light).

The measuring space may be formed by the first measuring chamber and/or the second measuring chamber. This may be understood to mean that the measuring space is formed by the first measuring chamber and the second measuring chamber, or only by the first measuring chamber, or only by the second measuring chamber. If the first and/or second measuring chamber(s) form(s) the measuring, the measurement is performed directly in the respective measuring chamber, i.e., the light beam generated by the light source (together with a transmitted component) passes through the respective measuring chamber for the purpose of the measurement, and is detected afterwards.

Alternatively, the measuring space may be arranged outside the measuring cell. In this case, the measuring space may be filled or prefilled with the first and/or second liquid measuring chamber medium. This may be understood to mean that the measuring space is filled with the medium of the first and second liquid measuring chambers. Alternatively, the measuring space may be filled only with the medium of the first liquid measuring chamber, or only with the medium of the second liquid measuring chamber. When the measuring space is arranged outside the measuring cell, it may be provided that the medium of the first and/or second liquid measuring chamber is introduced into the measuring space, through a liquid-transporting component, for example. Multiple external measuring spaces may also be provided. An external measuring space may be a cuvette, for example.

The first optical waveguide arranged between the light source and the measuring space may be configured to forward the light beam from the light source to the measuring space, and the second optical waveguide arranged between the measuring space and a detector may be configured to forward the light beam from the measuring space to the detector.

It is also conceivable to split the light beam that is generated by the light source into sub-beams before it enters the first and second measuring chambers, by means of a beam splitter, for example. Alternatively, multiple light sources may also be provided for generating multiple light beams, which may then be guided into the respective measuring spaces. In both cases, it may be provided that either a specific sub-beam or a light beam generated by a specific light source is used to carry out a zero measurement or a calibration measurement in one of the measuring chambers (or a measuring space or measuring spaces).

The detector may be designed to detect a transmitted component of the light beam after the light beam has passed through the medium of the first and/or second liquid measuring chamber present in the measuring space.

The concentration of a light-absorbing substance in solution may be determined using the logarithmised intensity of light that a solution (e.g., a liquid measuring chamber medium) absorbs at a certain wavelength, its absorbance. The relationship between absorbance Aλ at wavelength λ and concentration c is represented by the Beer-Lambert-Beer law:

A_{λ}=ε₈₀ *c*d (equation 1)

The extinction coefficient ε₈₀ in this equation is a wavelength- and substance-specific constant, and d is the distance the light travels through the sample (e.g., the measuring space).

The evaluation unit may be designed to perform a qualitative and/or quantitative determination of the cations and/or anions present in the ion-containing liquid medium on the basis of a signal received from the detector. This may be understood to mean that the evaluation unit is designed to determine both anions and cations, or only anions, or only cations, wherein the determination may be performed both qualitatively and quantitatively, or only qualitatively, or only quantitatively, in each case. In this context, anions and/or cations may first be determined quantitatively and/or qualitatively in the respective measuring chamber media. Then, on this basis, qualitative and/or quantitative conclusions may be drawn regarding anions and/or cations in the ion-containing liquid medium. The evaluation unit may be understood to be an arithmetic unit or computer, or part of an arithmetic unit or computer. An arithmetic unit or computer may be fixed in position or portable. The evaluation unit may be arranged outside the detector, in which case the detector and the evaluation unit are connected via suitable signal communication interfaces. The measuring arrangement (and thus the evaluation unit as well) is preferably computer-assisted, e.g., operated with the use of said arithmetic unit or said computer. To ensure its operation, the measuring arrangement (and thus the evaluation unit as well) has recourse to a software program, a routine, an algorithm or the like, which are executable on the arithmetic unit or computer. The measuring arrangement, but particularly the evaluation unit, may be operated on the basis of artificial intelligence (AI), for example AI algorithms or corresponding (teachable) neural networks. Said arithmetic unit or computer may include any and all necessary interfaces, power supply and storage units, data memories etc. that are needed for operating the measuring arrangement. Said arithmetic unit or computer may also comprise an open- or closed-loop control unit, with which further components of the analysis systems can be controlled in an open- or closed-loop manner.

According to a further variant of the invention, the light source, the detector and the evaluation unit (i.e. the measuring arrangement) are arranged outside the measuring cell. Alternatively, the light source, the detector and the evaluation unit (i.e. the measuring arrangement) are accommodated together in a shared unit (e.g., a shared housing). In the case of an external arrangement, the detector, the evaluation unit and the light source may also be arranged in a shared housing. Optionally, all components may be operated by a shared arithmetic unit. The housing is preferably made from a lightweight material, e.g., plastic.

According to a further variant of the invention, the sample chamber has an inlet for receiving the ion-containing liquid medium. An inlet may have one or more openings, through which the ion-containing liquid medium is can be introduced for filling the sample chamber. Other liquids, e.g., flushing fluids, may also be introduced into the sample chamber via such an inlet. The one or more opening(s) may be closable (by suitable closing means). The inlet may be fitted with a seal. The same applies similarly for an outlet from the sample chamber, which may also have one or more openings. The one or more opening(s) of the outlet may also be closable. Ion-containing liquid medium may be discharged from the sample chamber and thus also from the measuring unit via the outlet.

Accordingly, the sample chamber may either be filled with the ion-containing liquid medium through the inlet, or the ion-containing liquid medium may flow through the sample chamber from the inlet to an outlet. If the ion-containing liquid medium flows through the sample chamber from the inlet to an outlet, the analysis system may further comprise a sample reservoir for the ion-containing liquid medium which is connected to the inlet of the sample chamber via a liquid-carrying component, and a pump unit which is designed to pump the ion-containing liquid medium through the sample chamber from the sample reservoir and via the liquid-carrying component.

According to a further variant of the invention, the analysis system may comprise a filter unit arranged between the pump unit and the inlet to filter the ion-containing liquid medium. In this context, the ion-containing liquid medium may be guided through a filter arranged in the filter unit, so that substances that are harmful to the membrane can be trapped by the filter and so be removed from the ion-containing liquid medium. The provision of a filter unit is particularly advantageous for the stability and selectivity of membranes, for example cation- or anion-selective membranes, which are susceptible to corrosion or clogging by substances that are harmful to the membranes, particularly when under continuous stress. In the simplest case, a filter unit may be understood to be a strainer. Suitable filter materials may also be used, provided they also ensure that the substances that harmful to membranes referred to above can be filtered out (e.g., filter paper, molecular sieve, silica, activated charcoal, filter ceramics, nanopore filter etc.).

According to a further variant of the invention, the electrodes arranged in the measuring chambers may be made of metal. In this case, the metal from which the first electrode arranged in the first measuring chamber is made may be the same metal from which the second electrode in the second measuring chamber is made. Alternatively, the first electrode arranged in the first measuring chamber and the second electrode arranged in the second measuring chamber may be made from different metals and have different electrochemical potentials. In the context of the invention, for example, electrodes made from titanium or platinum may be used. Preferably, both the first electrode in the first measuring chamber and the second electrode in the second measuring chamber are made from titanium or platinum. Particularly preferably, the first electrode in the first measuring chamber and the second electrode in the second measuring chamber are each made from a titanium sheet or platinum sheet.

As noted earlier, the first liquid measuring chamber medium present in the first measuring chamber may contain a first detection substance for forming anion detection substance associates when anions pass into the first measuring chamber, and the second liquid measuring chamber medium present in the second measuring chamber may contain a second detection substance for forming cation detection substance associates when cations pass into the second measuring chamber. Accordingly, the invention is not limited to a certain detection substance for the purpose of forming associates, but rather all detection substances that are known and suitable for forming associates for the respective ions to be determined may be used as part of the invention.

In this context, a detection substance may be understood to refer to a chemical substance which is suitable for detecting ions in a liquid medium. In such a case, the detection substance may typically be used specifically for the detection of certain ions. In other words, a specific detection substance is usually suitable for detecting specific ions, i.e., specific cations or anions. For the purposes of the present invention, in principle all known detection substances may be used for the detection of ions.

In the context of the present invention, the detection of the ions by the detection substance may be because the detection substance forms ion detection substance associates with ions in a liquid medium, with the result that a detectable chemical or physical state of the liquid medium changes. The detectable chemical or physical state change may consist in that the pH, the colour, the temperature, or the light permeability of the liquid ion-containing medium changes, in isolation or in combination. In view of the above, the detectability of the chemical or physical change of state may be understood to consist in that the change of the chemical or physical state may be detected with the aid of a measurement, for example a photometric measurement.

In the context of the present invention, the detection substances may be for example complex indicators or metal indicators, and accordingly the ion detection substance associates may be metal cation-metal indicator complexes. In this context, metal indicators may be understood to be detection substances that form reversible complexes with metal ions. Accordingly, reversible complexes may be understood to be chemical compounds that consist of a coordination centre or central ion such as a metal ion, and ligands, for example chelating ligands. Chelating ligands may be understood to refer to ligands which are attached to a central ion by several coordination points, to form a chelating complex. The chemical bond between the coordination centre and the ligand may be understood to be a coordinating chemical bond, starting from the ligand, which transfers electron density to the coordination centre via free electron pairs. Since the coordinate chemical bond is considerably weaker than a covalent chemical bond, the formation of the complex is a reversible process, in which the reversible complex and the attached ligands and the unattached central ion exist in a dynamic equilibrium. In this situation, metal indicators represent a particularly suitable form of chelate complexing agents, wherein the reversible metal-indicator complex formed from metal ion and metal indicator has a different detectable colour from the pure, unbound metal indicator. By virtue of their structure, metal indicators are detection substances can change to a detectable degree not only the colour but also the pH of a liquid medium containing metal ions to be investigated and the metal indicator or metal-indicator complexes formed therefrom. The metal indicator may be a dye such as an azo dye, for example. The metal indicator may be represented for example by 2-Hydroxy-5-methyl-benzolazo-1-(2-naphthol-4-sulfonic acid) (CAS number 3147-14-6, C₁₇H₁₄N₂O₅S), available commercially under the trade name Calmagite.

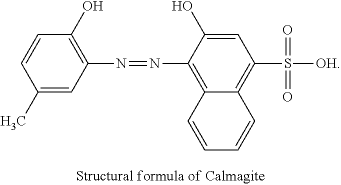 [SMILES: Cc1ccc(O)c(N=Nc2c(O)cc(S(=O)(=O)O)c3ccccc23)c1]

Especially because of its high chemical stability even in aqueous solution, Calmagite is a particularly suitable detection substance. Calmagite is provided in a liquid ion-containing medium such as an aqueous solution, in a simply to triply deprotonated form depending on the pH of the medium. It is the doubly deprotonated form that functions as metal indicator. Calmagite may be used for example for the determination of divalent cations such as calcium cations or magnesium cations, trivalent cations such as aluminium cations.

In the context of the present invention, the detection substances may further be precipitants, and the ion detection substance associates may accordingly be ion precipitant associates. In this respect, a precipitant may be understood to be a chemical compound which forms ion precipitant associates with ions present in a liquid medium, which associates are insoluble in said medium and are consequently precipitated out of the medium. Precipitation of an ion from a liquid medium such as an aqueous solution by a precipitant may also be understood to refer to process by which a previously dissolved ion is separated out in the form of a precipitate consisting of ion precipitant associates. In the context of the present invention, all known precipitants may be used.

Due to the formation of ion precipitant associates that are insoluble in the liquid medium, the light permeability of the medium may be reduced detectably. The precipitant may be for example sodium tetraphenylborate (Na[B(C₆H₅)₄], CAS number 143-66-8), available commercially under the trade name Kalignost. Kalignost is particularly suitable for detecting potassium, ammonium, rubidium, caesium, and thallium ions.

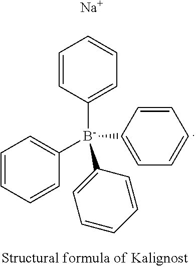

According to a variant of the invention, the first detection substance and/or the second detection substance may be chosen from the group of Calmagite, dimethylglyoxime, calcon carboxylic acid, xylenol orange, eriochrome black T, eriochrome blue black R, ethylenediaminetetraacetic acid (EDTA), oxalic acid, Kalignost, murexide, methylthymol blue, metal phthalein, pyrocatechol violet, 1-(2-Pyridylazo)-2-naphthol, 4-(2-Pyridylazo)resorcinol, iron(III)chloride, or mixtures thereof. This may be understood to mean that both the first and the first detection substance, or only the first detection substance, or only the second detection substance is chosen from the group of Calmagite, dimethylglyoxime, calcon carboxylic acid, xylenol orange, eriochrome black T, eriochrome blue black R, ethylenediaminetetraacetic acid (EDTA), oxalic acid, Kalignost, murexide, methylthymol blue, metal phthalein, pyrocatechol violet, 1-(2-Pyridylazo)-2-naphthol, 4-(2-Pyridylazo)resorcinol, iron(III)chloride, or mixtures thereof. Preferably, both the first detection substance and the second detection substance are chosen from the group of Calmagite, dimethylglyoxime, calcon carboxylic acid, xylenol orange, eriochrome black T, eriochrome blue black R, ethylenediaminetetraacetic acid (EDTA), oxalic acid, Kalignost, murexide, methylthymol blue, metal phthalein, pyrocatechol violet, 1-(2-Pyridylazo)-2-naphthol, 4-(2-Pyridylazo)resorcinol, iron(III)chloride, or mixtures thereof. The second detection substance is particularly preferably Calmagite.

When it is performed with the analysis system according to the invention, the use of various detection substances the analysis process according to the invention can be adapted to the conditions of a range of application areas without great effort, and without the need to make any technical changes to the analysis system for this purpose. The selection of suitable detection substances, a suitable metal indicator for example, also lends insensitivity to interfering ions.

Within the scope of the invention, all known ions that may be contained in the ion-containing liquid can be determined. In this context, ions may be understood to be electrically charged particles that are created from uncharged atoms or molecules by losing or gaining electrons. Anions and cations can be monovalently or polyvalently charged.

Cations may generally be understood to be positively charged ions which are formed from uncharged atoms or molecules by losing electrons. On the other hand, anions may be understood to be negatively charged ions which are formed from uncharged atoms or molecules by gaining electrons. When a voltage is applied between two electrodes, causing one electrode to be negatively polarised and the other to be positively polarised, cations dissolved in a liquid medium are attracted to the negatively charged electrode due to their positive charge, while anions are attracted to the positively charged electrode due to their negative charge.

According to a variant of the invention, cations present in the ion-containing liquid medium, such as iron(II) ions, iron(III) ions, copper(II) ions, magnesium(II) ions, calcium(II) ions, manganese(II) ions, potassium(I) ions, ammonium cations (NH₄+), or mixtures thereof, can be determined. Alternatively, or in addition thereto, anions present in the ion-containing liquid medium may be nitrate ions (NO₃−), nitrite ions (NO₂−), phosphate ions (PO₄³−), or mixtures thereof. The cations present in the ion-containing liquid medium that are determined are preferably iron(II) ions, iron(III) ions, copper(II) ions, magnesium(II) ions, calcium(II) ions, manganese(II) ions, potassium(I) ions, ammonium cations (NH4+), or mixtures thereof, and the anions present in the ion-containing liquid medium that are determined are preferably nitrate ions (NO₃−), nitrite ions (NO₂−), phosphate ions (PO₄³−), or mixtures thereof.

Additionally, the first and/or second liquid measuring chamber medium may contain further complexing agents. In this situation, a further complexing agent may be any chemical substance which is able to mask interfering substances contained in the measuring chamber media. Masking of interfering substances may be understood to mean complexing of the interfering substances by the further complexing agent, with the result that the interfering substances are converted into masked substances. Mask substances may be understood to be substances which are no longer able to interfere with the analysis process according to the invention, in particular photometric measurement in the measuring chambers. Also, the further complexing agent can be regenerated in a similar way to the regeneration of detection substances described herein.

According to a further variant of the invention, the first and/or second liquid measuring chamber medium may contain an electrolyte to increase conductivity. In the context of the present invention, an electrolyte may be understood to be a chemical substance which is added to the first and/or second liquid measuring chamber medium and is designed to increase the conductivity of the respective measuring chamber medium. In such a case, when it is added to a measuring chamber the electrolyte may dissociate into its ions, and when a voltage is applied between electrodes arranged in the measuring chambers it may direct the electrical current under the influence of the electrical field produced thereby, which increases the conductivity of the measuring chamber medium compared with a measuring chamber medium that does not contain an electrolyte. In this context, conductivity is described as the ability to direct an electrical current. In general, all known electrolytes may be used. The electrolyte is particularly preferably sodium sulfate.

Alternatively, an ion exchanger material may be arranged in the sample chamber to increase the conductivity of the liquid medium. In this context, the liquid medium may flow through the ion exchanger material arranged in the sample chamber, wherein ions of one ion type are bound to the ion exchanger material, and an equivalent charge quantity of ions of another ion type previously bound to the ion exchanger material is released into the medium, thus increasing the conductivity of the liquid medium. In this context, an ion exchanger material may be understood to be a material that is able to exchange one ion type that was originally present in the liquid medium for another ion type that was originally bound to the ion exchanger material. The ion exchanger material may be for example aluminium oxide, chlorophyll, colestyramine, colesevelam, a synthetic resin based on at least one polymer material, or a zeolite such as zeolite A (Sasil).

The ion exchanger material may be a cation exchanger material, an anion exchanger material, or an amphoteric ion exchanger material. In this context, a cation exchanger material can bind a cation type which is dissolved in the liquid ion-containing medium and release a different cation type which was originally bound to the ion exchanger material into the medium, so that in the course of the exchange process one cation type is exchanged for the other cation type. Similarly, an anion exchanger material can exchange anions of one anion type for anions of a different anion type. Further, an amphoteric ion exchanger material can exchange anions and cations at the same time.

During this process, cation exchanger materials contain anionic functional group on their surface, such as carboxylic acid or sulfonic acid groups, with dissociable cations. Anion exchangers in turn contain cationic functional groups, such as quaternary ammonium groups, which can exchange their counteranions.

According to a further variant of the invention, the anion-selective membrane and/or the cation-selective membrane may each be made from a membrane material that includes a first and/or a second polymer material. The first polymer material contains functional groups arranged on a polymer material surface which provide anion selectivity of the anion-selective membrane and/or cation selectivity of the cation-selective membrane. In general, functional groups suitable for providing an anion selectivity of an anion-selective membrane are those that are positively polarisable, thereby enabling anions to pass through the membrane, but repelling cations and thus preventing them from passing through the membrane. Similarly, functional groups suitable for providing a cation selectivity of a cation-selective membrane are those that are negatively polarisable, thereby enabling cations to pass through the membrane, but repelling anions and thus preventing them from passing through the membrane.

The first polymer material is preferably modified polystyrene (PS) with functional groups arranged on a polymer material surface. The functional groups arranged on the polymer material surface for assuring the anion selectivity of the anion-selective membrane are generally positively polarisable functional groups, preferably quaternary amines. Alternatively, or additionally, the functional groups arranged on the polymer material surface for assuring the cation selectivity of the cation-selective membrane are negatively polarisable functional groups, preferably carboxylic acid or sulfonic acid groups.

The membrane material may further comprise a second polymer material for the mechanical reinforcement of the corresponding anion-selective membrane and/or the cation-selective membrane. Here, mechanical reinforcement of a membrane may be understood to be greater resistance of the membrane to mechanical influences, such as a mechanical impact, which may be achieved by building a second polymer material into the membrane. The second polymer material then comprises at least one polymer suitable for mechanical reinforcement. The second polymer material is preferably polyvinyl chloride (PVC), polypropylene (PP) or polyethylene terephthalate (PET).

According to a further variant of the invention, it may be provided that the analysis system comprises a plurality of first measuring chambers and/or second measuring chamber. Several pairs of first and second measuring chambers may be provided. Sample chambers may be arranged between the respective pairs, so that in total a plurality of measuring cells may be provided, each consisting of a first measuring chamber, a second measuring chamber and a sample chamber arranged between each of them. Multiple measuring cells may be advantageous for making it possible to investigate different ion types qualitatively and/or quantitatively at the same time or for assuring different measuring conditions (e.g., different detection substances etc.).

According to a further variant of the invention, the analysis system as a compact probe with integrated pump unit may be arranged directly in a sample reservoir. In this context, a sample reservoir may be understood to be a static or flowing medium for analysis, for example a body of standing water or flowing water, such as a lake or a river, or a standing or flowing industrial medium, such as a flow reactor. Sewage plants, water treatment plants etc. may also represent standing or flowing media which can be investigated with the analysis system described. Similarly, an aquarium filled with water may serve as a sample reservoir. The water contained in an aquarium may constitute the static or flowing water to be analysed. One of the advantages of an analysis system in the form of a compact probe with integrated pump unit is that the compact probe can be manufactured and maintained very easily and inexpensively, and can be placed directly in a sample reservoir, so that very precise measurement values can be obtained, also in the course of a real-time measurement such as an online or inline measurement.

Alternatively, the analysis system may also be embodied as a compact probe without a pump unit. In this case, the analysis system may be arranged directly in a sample reservoir with a flowing medium (e.g., flowing body of water) with a flow velocity. In this case, because of the flow velocity of the flowing medium, it is not necessary to pump the ion-containing liquid medium through the sample chamber, and the provision of a pump unit can be dispensed with. Such an analysis system is advantageous because it can be manufactured, used, and maintained easily and inexpensively. In this case too, the sample reservoir may be an aquarium.

Alternatively, the analysis system may be arranged externally to a sample reservoir (with a flowing or standing medium to be analysed). Then, the sample chamber is filled with ion-containing liquid medium from the reservoir. Ion-containing liquid medium may also be fed (e.g., pumped) from such a reservoir into the sample chamber. In this case too, the sample reservoir may be an aquarium.

As was noted earlier, the problem that the present invention is intended to address is also solved with an analysis process according to claim 13. Said analysis process comprises at least the following process steps S2 and S3:
S2: photometric measurement of anions, anion detection substance associates, cations and/or cation detection substance associates with the measuring arrangement,
S3: qualitative and/or quantitative determination of the anions and/or cations based on the photometric measurement in step S2.

Process step S2 relates to
photometric measurement of anions and/or anion detection substance associates present in the first measuring chamber with the measuring arrangement, and/or
photometric measurement of cations and/or cation detection substance associates present in the second measuring chamber with the measuring arrangement.

Process step S3 then relates to qualitative and/or quantitative determination of the anions and/or cations based on the photometric measurement in step S2. Step S3 may be carried out with an evaluation unit, which has been described several times earlier.

This may be explained with reference to the following three alternatives.

In a first alternative, in step S2 the anions and/or anion detection substance associates present in the first measuring chamber may be measured photometrically with the measuring arrangement by way of a photometric measurement. In this way, anions and anion detection substance associates may be measured photometrically. Alternatively, only anions, or only anion detection substance associates may be measured photometrically.

In step S3, the anions are then determined qualitatively and/or quantitatively based on the respective photometric measurement(s) in step S2. This may be understood to mean that a qualitative and a quantitative determination of the anions is performed. Alternatively, only a quantitative determination, or only a qualitative determination of the anions may be carried out. The qualitative and/or quantitative determination may be performed with an evaluation unit which has been described several times earlier.

Alternatively, in step S2 the cations and/or cation detection substance associates present in the second measuring chamber may be measured photometrically with the measuring arrangement by way of a photometric measurement. This may be understood to mean that cations and cation detection substance associates are measured photometrically. Alternatively, only cations, or only cation detection substance associates may be measured photometrically. In step S3, the cations are then determined qualitatively and/or quantitatively based on the respective photometric measurement(s) in step S2. This may be understood to mean that a qualitative and a quantitative determination of the cations is performed. Alternatively, only a quantitative determination, or only a qualitative determination of the cations may be carried out. The qualitative and/or quantitative determination may be performed with an evaluation unit which has been described several times earlier.

Further, with the measuring arrangement in step S2, both the anions and/or anion detection substance associates present in the first measuring chamber, and the cations and/or cation detection substance associates present in the second measuring chamber may be measured. In step S3, the anions and the cations are then each determined qualitatively and/or quantitatively based on the respective photometric measurement(s) in step S2. This may be understood to mean that the qualitative and the quantitative determination of the anions and cations takes place at the same time. Alternatively, the anions and the cations may be determined only quantitatively, or only qualitatively. The qualitative and/or quantitative determination may be performed with an evaluation unit which has been described several times earlier.

A photometric measurement may be represented by a measuring process in which liquid media may be analysed in terms of their composition, for example regarding ions present therein, with the aid of a light source. Thus, in a photometric analysis for example, a chemical substance may be analysed using its specific colour reaction and light absorption as a function of the chemical property at a specific wavelength. In this context, for a photometric measurement a light beam might be produced by a light source and passed through a measuring space. The light beam may then exit the measuring space again as a transmitted component of the light beam and then detected by a detector. In such a case, transmitted component of the light beam may be understood to be a light beam with reduced light intensity compared with the light beam that was directed into the measuring space. The reduction in light intensity may be attributed to interactions such as absorption, diffusion, refraction, or reflection of the light beam, with the medium located in the measuring space, which contains the ions that are to be investigated. In this way, the transmission (extinction, absorbance) may be understood as a measure of the light permeability of the ion-containing medium that is to be investigated, which is described with the following equation:

E=log I₀/I_{D}=log 1/D (Equation 2)
where I₀: Intensity of the light beam before passing through the measuring space,
I_{D}: Intensity of the light beam after passing through the measuring space,
Light permeability I_{D}/I₀.

The photometric analysis (which may be carried out with the measuring arrangement described) is notable for its speed, high selectivity, and low time requirement. It is of particular significance for the precise recording of low ion contents in a liquid medium, for the determination of metals in the trace range, for example. The light beam generated by the light source is preferably based on ultraviolet (UV) or visible (VIS) light. In a photometric measurement, the transmission of a fluid medium may then be measured as a function of the wavelength of the irradiated light, and a transmission spectrum obtained therefrom. The transmission spectrum may then be used to determine the ions qualitatively based on characteristic phenomena in the transmission spectrum, and/or quantitatively with the aid of suitable calculation methods. Characteristic phenomena of the transmission spectrum may be characteristic curve features, for example peaks, maxima, minima, inflection points, slopes, smoothness, curvature, wavelength-dependent positions (or shifts) of the abovementioned features or distances from characteristic curve features in the spectrum. Relationships between characteristic curve features (e.g., relationships between averaged intensities or the like) may also be considered in the evaluation. In particular, the qualitative and/or quantitative determination may be carried out with an evaluation unit, as has been described several times previously.

A further, optional step S1 also concerns,
Forming anion detection substance associates from anions present in the first measuring chamber and the first detection substance, and/or
Forming cation detection substance associates from cations present in the second measuring chamber and the second detection substance.

The subordinate claims relate to advantageous variants and further developments of the present invention. The features described in the subordinate claims may be implemented in any combination to further develop the analysis process according to the invention, if such development is technically feasible. This still applies if combinations of such kind are not illustrated explicitly with corresponding references in the claims. This also remains applicable beyond the category limits of the claims.

According to an advantageous variant of the process according to the invention, a step S0-a may be performed prior to optional step S1, or prior to step S2. Step S0-a concerns an application of a voltage between the first electrode and the second electrode, which has the effect of polarising the first electrode positively and the second electrode negatively, and causes anions to migrate from the sample chamber, through the anion-selective membrane towards the first electrode arranged in the first measuring chamber, and cations to migrate from the sample chamber, through the cation-selective membrane towards the second electrode arranged in the second measuring cell.

According to a further advantageous variant of the analysis process according to the invention, a step S0-b may be performed prior to optional step S1, or prior to step S2. Step S0-b relates to an adjustment of a pH of the first liquid measuring chamber medium contained in the first measuring chamber and/or of the second liquid measuring chamber medium contained in the second measuring chamber with an analysis system according to claim 3.

According to a further advantageous variant of the analysis process according to the invention, a step S4 may be performed after step S3 or prior to step S0-a. Step S4 relates to an application of a voltage between the first electrode and the second electrode that is reversed with respect to the voltage applied in S0-a, which polarises the first electrode negatively and the second electrode positively.

The effect of this is that:
the anion detection substance-associates formed in the first measuring chamber in optional step S1 are reverted to anions and the first detection substance, and anions are transported back from the first measuring chamber, through the anion-selective membrane and into the sample chamber, whereby the first detection substance is regenerated, and/or
the cation detection substance associates formed in the second measuring chamber in optional step S1 are reverted to cations and the second detection substance, and cations are transported back from the second measuring chamber, through the cation selective membrane and into the sample chamber, whereby the second detection substance is regenerated.

In principle, all process steps relating to measurement and/or evaluation may be carried out with computer support, i.e., with the use of a suitable arithmetic unit. Such an arithmetic unit may rely on computing routines, algorithms, software etc. As noted earlier, such an arithmetic unit may be a computer. An arithmetic unit may be programmable.

Further advantages, variants and further developments relating to the analysis system according to the invention and/or the analysis process according to the invention, are explained in greater detail with reference to the embodiments described in the following text. These are intended to explain the invention to the person skilled in the art and enable him to carry out the invention but without limiting the invention. In conjunction with the above description of the embodiments, reference is made to the following figures, which serve as the basis for a more complete description of the analysis system and the analysis process according to the invention. In the drawing:

### BRIEF DESCRIPTION OF THE DRAWINGS

FIG. 1 is a schematic representation of a measuring cell which is used in an embodiment of the analysis system according to the invention;

FIG. 2 is a schematic representation of an analysis system according to an embodiment of the invention;

FIG. 3 is an exemplary absorption spectrum of calcon carboxylic acid as an example of a detection substance;

FIG. 4 is an exemplary absorption spectrum of Calmagite as a further example of a detection substance; and,

FIG. 5 is an absorption spectra of various metal indicators together with various ions for determination.

### DETAILED DESCRIPTION OF THE DRAWINGS

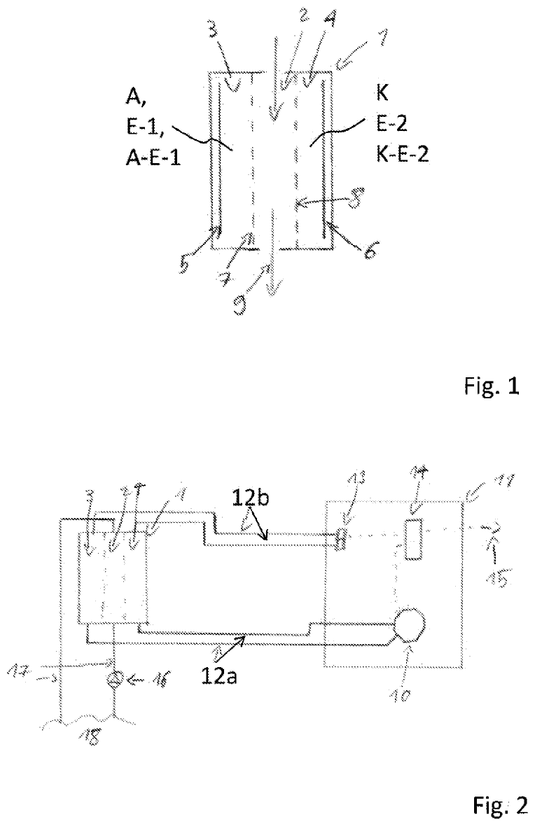

FIG. 1 shows a measuring cell 1 which is used in an analysis system according to the invention according to FIG. 2. The analysis system is designed to carry out qualitative and/or quantitative determination of ions in an ion-containing liquid medium 9. The ions may be cations K or anions A. As shown in FIG. 1, the measuring cell comprises a first measuring chamber 3, a second measuring chamber 4, and a sample chamber 2 arranged between the first measuring chamber 3 and the second measuring chamber 4, wherein the sample chamber 2 is designed to accommodate the ion-containing liquid medium 9. FIG. 1 shows an example of a sample chamber 2 through which an ion-containing liquid medium 9 flows. A first electrode 5 is arranged in the first measuring chamber 3, and a second electrode 6 is arranged in the second measuring chamber 4. A voltage may be applied between the first and second electrodes 5, 6, whereby the first electrode 5 may be positively polarised and the second electrode 6 negatively polarised, and consequently the anions A may be transported towards the first electrode 5 and cations K transported towards the second electrode 6. An anion-selective membrane 7 is arranged between the first measuring chamber 3 and the sample chamber 2, and a cation-selective membrane 8 is arranged between the second measuring chamber 4 and the sample chamber 2. The anion-selective membrane 7 is designed to enable a passage of anions A out of the sample chamber 2 into the first measuring chamber 3. The cation-selective membrane 8 is designed to enable a passage of cations K out of the sample chamber 2 into the second measuring chamber 4. The first measuring chamber 3 is filled with a first liquid measuring chamber medium. The first liquid measuring chamber medium may be the ion-containing liquid medium 9. The first liquid measuring chamber medium optionally contains a first detection substance E-1, to form anion-detection substance associates A-E-1 when the anions A pass into the first measuring chamber 3. The second measuring chamber 4 is filled with a second liquid measuring chamber medium. The second liquid measuring chamber medium optionally contains a second detection substance E-2, to form cation detection substance associates K-E-2 when the cations K pass into the second measuring chamber 4.

FIG. 2 shows the measuring cell of FIG. 1 in highly schematic form, and a measuring arrangement. The measuring arrangement is designed to measure the anions A and/or anion detection substance associates A-E-1 present in the first measuring chamber 3 photometrically by way of a photometric measurement, and to determine the anions A qualitatively and/or quantitatively based on this/these measurement(s). Alternatively, or additionally thereto, the measuring arrangement is designed to measure the cations K and/or cation detection substance associates K-E-2 present in the second measuring chamber 4 photometrically by way of a photometric measurement, and to determine the cations K qualitatively and/or quantitatively on the basis of this/these measurement(s).

As is represented in the figures, the measuring arrangement comprises a light source 10, which is designed to produce a light beam. The measuring arrangement further comprises a measuring space, wherein the measuring space according to the present example is formed by the first measuring chamber 3 and the second measuring chamber 4.

A first optical waveguide 12a is arranged between the light source 10 on the one hand and the first measuring chamber 3 and the second measuring chamber 4 on the other (these function as measuring spaces), and is configured to guide the light beam from the light source 10 to the measuring space (i.e., the first measuring chamber 3 and second measuring chamber 4). A second optical waveguide 12b is arranged between the first measuring chamber 3 and second measuring chamber 4 on the one hand and a detector 13 on the other, and is configured to guide the light beam from the first measuring chamber 3 and the second measuring chamber 4 to the detector 13, wherein the detector 13 is designed to detect a transmitted component of the light beam after the light beam has passed through a first or second liquid measuring chamber medium located in the measuring space. An evaluation unit 14 is also provided and is designed to determine the cations and/or anions present in the ion-containing liquid medium 9 qualitatively and/or quantitatively based on a signal received from the detector 13. The figure shows that light source 10, the detector 13 and the evaluation unit 14 arranged in an assembly (e.g., a housing 11) outside of the measuring cell 1. The evaluation unit 14 is operated by a user 15, who may program or read out data therefrom.

FIG. 2 further shows that ion-containing liquid medium 9 is transported out of a sample reservoir 18 and through a liquid-carrying component 17 (e.g., a line, pipe, hose or the like) and into the sample chamber 2. The ion-containing liquid medium is also removed from the sample chamber 2 again, for example by use of a pump unit 16.

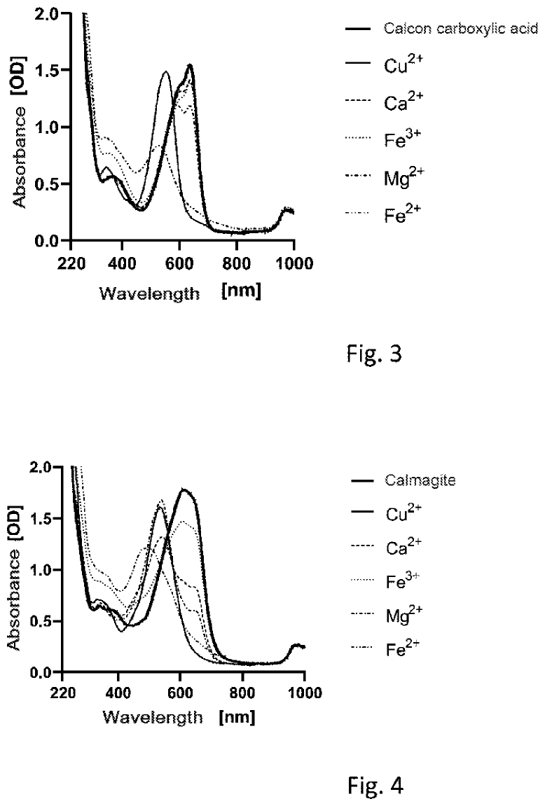

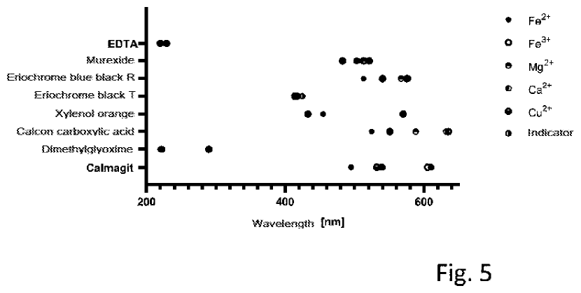

FIG. 5 shows the position of the absorption maxima of a variety of metal cation-metal indicator complexes according to corresponding photometric measurement. It is to be expected that the metal cations are clearly differentiated when the absorption maxima are as far as possible from each other and do not overlap. This is the case particularly with Calmagite and calcon carboxylic acid, which is why in FIGS. 3 and 4 corresponding absorption spectra for calcon carboxylic acid alone (FIG. 3) and Calmagite alone (FIG. 4) are shown compared with the absorption spectra of calcon carboxylic acid ion associates (with the Cu²⁺, Ca²⁺, Fe³⁺, Mg²⁺ and Fe²⁺) in FIG. 3 and calmagite ion associates (with the ions Cu²+, Ca²+, Fe³+, Mg²⁺ and Fe²⁺) in FIG. 4. The measurements were carried out with concentrations of calcon carboxylic acid and Calmagite and ion concentrations of 0.0001M in each case (except Ca²⁺, 0.001 M). The pH was adjusted to pH=10 with a 0.5 M ammonium/ammonia buffer solution. FIGS. 3 and 4 reveal that the absorption curves of the detection substance ions associates are shifted compared to the pure detection substances, and the respective absorption curves have different curve features. The cations listed can be differentiated qualitatively on this basis alone. This may also be done for example by comparing the respective curves with database or literature data.

In the following, an example of an option for the quantitative determination of cations (in this case Cu²⁺) with an analysis system and analysis process according to the invention using Calgamite as the detection substance is described with reference to an exemplary calculation/derivation.

The “Rank Annihilation Factor Analysis (RAFA)” method for the analysis of spectroscopic data such as that of UV-Vis spectroscopy is known from the prior art. Thus, for example, metal indicator (detection substances) can be analysed more precisely, and their complexing constants with a specific metal, but also the extinction coefficients of the metal-indicator complexes formed that cannot be accessed directly by experimental means can be determined. In this example, a model first had to be created which calculates the absorbance of samples with specific initial concentrations of indicator and metal salt from the two cited constants (complexing constant, extinction coefficient). Then, real samples are measured with the concentrations used in the model. The constants used in the model are varied, and the result from the model is compared with the real data. The point with the smallest relative standard deviation between sample and model reflects the optimum for the complexing constant and the extinction coefficient in this example.

Ion flow in systems such as the one described here can be described with the Nernst-Planck equation:

Jᵢ=−Dᵢ*∇_{ci}−zᵢ*uᵢF*cᵢ*∇_{ϕ} (Equation 3)

Here, Jᵢ describes the flow of ions i at this location. Dᵢ is the coefficient of diffusion, ci is the concentration at the location under consideration, zᵢ is the charge number, and uᵢ is the mobility of i, φ describes the electrical potential. The first term here models the diffusion, and second models the flow of ions due to the voltage applied in the cell.

For the determination of the extinction coefficient of the metal-indicator complexes used and their complexing constants, an RAFA-like method can be used. For these purposes, it is initially assumed that only the following reaction takes place between metal ion (M²⁺) and indicator (Ind²⁻):

M²⁺+Ind²⁻ [symbol] [M(Ind)]

The complex formed is abbreviated to M-Ind in the following text. For this reaction, the law of mass action can now be applied with the complexing constant K_{B}:

$$\\ \begin{matrix}{K}_{B} = \frac{[ M - Ind ]}{[ {M}^{{}_{} 2 +} ] * [ {Ind}^{{}_{} 2 -} ]} & ( Equation 4 )\end{matrix}$$

Additionally, the following dependencies on the original concentrations cᵢ apply for the concentrations in equilibrium:

[M²⁺]=c_{M2+}+−[M−Ind] (Equation 5)

[Ind²⁻]=c_{Ind2-}−[M−Ind] (Equation 6)

In the following, [M-Ind] is abbreviated to x. Inserting equations 5 and 6 in equation 4 and transposing yields equation 7:

|K_{B}*c_{Ind}_{^{2−}}*c_{M}_{²⁺}−(K_{B}*(c_{Ind}_{^{2−}}+c_{M}_{²⁺})+1)*x+K_{B}*x²=0 (Equation 7)

Equation 8 is obtained by resolving equation 7. In this case only the solution with a negative prefix before the root is meaningful.

$$\\ \begin{matrix}x = \frac{{K}_{B} * ( {c}_{{Ind}^{{}_{} 2 -}} + {c}_{{M}^{2 +}} ) + 1 - \sqrt{{( {K}_{B} * ( {c}_{{Ind}^{{}_{} 2 -}} + {c}_{{M}^{2 +}} ) + 1 )}^{2} - 4 * {K}_{B}^{{}_{} 2} * {c}_{{Ind}^{{}_{} 2 -}} * {c}_{{M}^{2 +}}}}{2 * {K}_{B}} & ( Equation 8 )\end{matrix}$$

For the total absorbance A_{ges} of the sample, it is assumed that the following equation applies:

A_{ges}=A_{Ind2-}+A_{M2+}+A_{M-Ind}+A₀ (Equation 9)

Here, A₀ is the absorbance when measuring the pure solvent. When the Lambert-Beer law and equations 5 and 6 are applied, the following relationship is obtained:

A_{ges}=(c_{Ind}_{^{2−}}−x)*ε_{Ind}_{^{2−}}+(c_{M}_{²⁺}−x)*ε_{M}_{²⁺}+x*ε_{M-Ind}+A₀ (equation 10)

The length of the cuvette can be standardised to 1 cm with a pathlength correction, and this can then be omitted for the sake of simplicity. The extinction coefficients for the pure salt ε_{M2+} and for the pure indicator ε_{ind2-} can be determined directly from absorbances of pure solutions of these substances by using the Lambert-Beer law. A_{ges} in turn can be determined by measuring a real solution with c_{Ind}²₋ and c_{M}²₊. Since x can be found using equation 8, the inaccessible extinction coefficient of the metal-indicator complex ε_{M-Ind} can now be determined with this data and by transposing equation 10:

$$\\ \begin{matrix}{ε}_{M - Ind} = \frac{{A}_{ges} - ( {c}_{{Ind}^{{}_{} 2 -}} - x ) * {ε}_{{Ind}^{{}_{} 2 -}} - ( {c}_{{M}^{2 +}} - x ) * {ε}_{{M}^{2 +}} - {A}_{0}}{x} & ( Equation 11 )\end{matrix}$$

Now to determine K_{B} and ε_{M-Ind}, samples with various c_{Ind2-} and c_{M2+} can be measured. Then, K_{B} can be varied over a broad value range, and ε_{M-Ind} determined for each individual sample. For each K_{B} applied, the relative standard deviation σ_{R} under the ε_{M-Ind} can be determined with the following formula:

$$\\ \begin{matrix}{σ}_{R} = \sqrt{\frac{{\sum }_{i = 1}^{n} {( {ε}_{M - Ind , i} - )}^{2}}{n - 1}} * \frac{1}{} & ( Equation 12 )\end{matrix}$$

The optimum value for K_{B} is obtained for the smallest σ_{R}. The optimum value for ε_{M-Ind} at this point is determined from the average of the individual ε_{M-Ind} of the individual samples. For the analysis of metal-indicator complexes with two indicator ligands per metal ion, analogous equations apply, starting from the following law of mass action:

$$\\ \begin{matrix}{K}_{B} = \frac{[ M - 2 Ind ]}{[ {M}^{{}_{} 2 +} ] * {[ {Ind}^{{}_{} 2 -} ]}^{2}} & ( Equation 13 )\end{matrix}$$

The calculation of the metal ion concentrations in the measuring chamber (in this case the second measuring chamber for cations) is complicated by the fact that the absorbances of the unbound Calmagite and those of the metal-Calmagite complexes overlap. To solve this problem, equation 9 is extended, and it is assumed that the absorbance of metal ions which are not bound is very low.

A_{ges,λ}=A_{Ind}_{^{2−}}_{,λ}+ΣA_{M}_{ᵢ}_{-Ind,λ}+A_{0,λ} (Equation 14)

A_{ges,λ}=ε_{Ind}_{^{2−}}_{,λ}*[Ind²⁻]+Σε_{M}_{ᵢ}_{-Ind}*[Mᵢ−Ind]+A_{0,λ} (Equation 15)

Equation 15 is obtained by applying the Lambert-Beer law. In this case, the extinction coefficient of the indicator εInd²⁻, λ for wavelength λ is already known from the described measurements. The extinction coefficients of the metal-indicator complexes εM_{i-Ind,λ} can be found with the data from the analysis of the metal-indicator complexes since the concentrations of the individual substances in the solution being analysed are determined. Accordingly, equation 11 can be used for other wavelengths as well, and the concentrations used. Now to determine the individual concentrations in the measuring cell itself, the absorbance must be determined at i+1 wavelengths. Since the extinction coefficients of the substances present can be determined for all wavelengths, now i+1 equations are obtained for the same number of unknown concentrations. Solving this equation system yields the equilibrium concentrations. Now to determine the total concentrations of the metal ions in the system, equations 4 and 5 are used:

$$\\ \begin{matrix}{c}_{M , i} = [ {M}_{i} - Ind ] * ( 1 + \frac{1}{{K}_{B , i} * [ {Ind}^{{}_{} 2 -} ]} ) & ( Equation 16 )\end{matrix}$$

The concentration of the copper ions is calculated with a similar equation based on equation 13.

To model the concentration in the measuring, a number of assumptions must be made first. For example, it must be assumed that the ion diffusion through the membrane is negligible. Moreover, the formation of boundary layers should also be ignored initially. Then one arrives at the following simplified Nernst-Planck equation for the ion flow Jᵢ through the membrane:

Jᵢ=−zᵢ*u₁*F*cᵢ*∇ϕ (Equation 17)

Now it is assumed that the potential φ in the solution only varies linearly and that ∇ϕ is therefore constant. Rewriting as a velocity law yields the following equation:

$$\\ \begin{matrix}- \frac{{dc}_{a}}{dt} = k * {c}_{a} & ( Equation 18 )\end{matrix}$$

Here, k describes the constants that occur, ca describes the concentration of the respective metal ion outside of the measuring chamber, i.e., in front of the membrane.

Integration then yields the following equation:

∫_{c}_{_{a,o}}^{c}^{_{a,1}}c⁻¹*dc=−k*∫₀^{t}dt (Equation 19)

It follows that:

c_{a,1}=c_{a,0}*e^{-k*t} (Equation 20)

c_{a,1}−c_{α,0}=Δc_{α} (Equation 21)

The change in the measurable concentration in the measuring chamber Δc over the volume V of this, and over the volume of the external sample Va is also dependent on the change in the external concentration ca, since the ions migrate from the sample to the measuring chamber. Thus, the following equation is obtained:

$$\\ \begin{matrix}Δ {c}_{a} = - Δ c * \frac{V}{{V}_{a}} & ( Equation 22 )\end{matrix}$$

By inserting equations 21 and 22 in equation 20, and then transposition, a law is obtained for determining the initial external concentration when the change in the internal concentration is known, with which it is ultimately possible to determine the concentration of the sample:

$$\\ \begin{matrix}{c}_{a , 0} = \frac{Δ c * \frac{V}{{V}_{a}}}{1 - {e}^{- k * t}} & ( Equation 23 )\end{matrix}$$

1. Analysis system for the qualitative and/or quantitative determination of ions in an ion-containing liquid medium, wherein the ions are cations (K) and/or anions (A), the analysis system comprising:
a measuring cell (1) comprising: a first measuring chamber, a second measuring chamber, and a sample chamber arranged between the first measuring chamber and the second measuring chamber, wherein the sample chamber is designed to accommodate the ion-containing liquid medium,
wherein a first electrode is arranged in the first measuring chamber and a second electrode is arranged in the second measuring chamber, and wherein a voltage can be applied between the first and second electrodes, whereby the first electrode can be positively polarized and the second electrode can be negatively polarized, and whereby the anions (A) can be transported towards the first electrode and the cations (K) can be transported towards the second electrode,
wherein an anion-selective membrane is arranged between the first measuring chamber and the sample chamber, and a cation-selective membrane is arranged between the second measuring chamber and the sample chamber, wherein the anion-selective membrane is designed to enable a passage of anions (A) out of the sample chamber into the first measuring chamber, and wherein the cation-selective membrane is designed to enable a passage of cations (K) out of the sample chamber into the second measuring chamber,
wherein the first measuring chamber is filled with a first liquid measuring chamber medium and wherein optionally a first detection substance (E-1) is contained in the first liquid measuring chamber medium, in order to form anion detection substance associates (A-E-1) when anions (A) pass into the first measuring chamber, and
wherein the second measuring chamber is filled with a second liquid measuring chamber medium and wherein optionally a second detection substance (E-2) is contained in the second liquid measuring chamber medium, in order to form cation detection substance associates (K-E-2) when cations (K) pass into the second measuring chamber,
and
a measuring arrangement that is designed
a. to measure the anions (A) and/or anion detection substance associates (A-E-1) present in the first measuring chamber photometrically by way of a photometric measurement, and to determine the anions (A) qualitatively and/or quantitatively on the basis of this/these measurement(s), and/or
b. to measure the cations (K) and/or cation detection substance associates (K-E-2) present in the second measuring chamber photometrically by way of a photometric measurement, and to determine the cations (K) qualitatively and/or quantitatively on the basis of this/these measurement(s).

2. Analysis system according to claim 1, wherein a first mixing apparatus is arranged in the first measuring chamber to mix the first liquid measuring chamber medium contained in the first measuring chamber, and/or wherein a second mixing apparatus is arranged in the second measuring chamber to mix the second liquid measuring chamber medium contained in the second measuring chamber.

3. Analysis system according to claim 1, wherein in order to adjust a pH of the first liquid measuring chamber medium contained in the first measuring chamber and/or of the second liquid measuring chamber medium contained in the second measuring chamber,
a. a charge-permeable membrane is arranged between the first measuring chamber and the second measuring chamber to enable a charge to pass between the first measuring chamber and the second measuring chamber, or
b. a first charge-permeable membrane is arranged between the first measuring chamber and a first water electrolysis chamber fitted with a first water electrolysis electrode, to enable a charge to pass between the first measuring chamber and the first water electrolysis chamber, and/or a second charge-permeable membrane is arranged between the second measuring chamber and a second water electrolysis chamber fitted with a second water electrolysis electrode to enable a charge to pass between the second measuring chamber and the second water electrolysis chamber.

4. Analysis system according to claim 1, wherein the measuring arrangement comprises:
a light source that is designed to generate a light beam,
a measuring space, wherein the measuring space
a. is formed by the first measuring chamber and/or the second measuring chamber, or
b. is arranged outside the measuring cell, wherein the measuring space can be filled with the first and/or second liquid measuring chamber medium,
a first optical waveguide arranged between the light source and the measuring space, which is configured to guide the light beam from the light source the measuring space, and a second optical waveguide arranged between the measuring space and a detector, which is configured to guide the light beam from the measuring space to the detector, wherein the detector is designed to detect a transmitted component of the light beam after the light beam has passed through first or second liquid measuring chamber medium located in the measuring space, and
an evaluation unit, which is designed to determine cations and/or anions present in the ion-containing liquid medium qualitatively and/or quantitatively on the basis of a signal received from the detector.

5. Analysis system according to claim 4, wherein the light source, the detector and the evaluation unit are arranged outside the measuring cell.

6. Analysis system according to claim 1, wherein the sample chamber has an inlet for receiving the ion-containing liquid medium, wherein the sample chamber
a. can be filled with the ion-containing liquid medium through the inlet, or
b. the ion-containing liquid medium can flow through the sample chamber from the inlet towards an outlet, wherein the analysis system further comprises:
a sample reservoir for the ion-containing liquid medium, which is connected to the inlet of the sample chamber via a liquid carrying component,
a pump unit which is designed to pump the ion-containing liquid medium out of the sample reservoir and through the sample chamber via the liquid-carrying component.

7. Analysis system according to claim 6, further comprising a filter unit arranged between the pump unit and the inlet in order to filter the ion-containing liquid medium.

8. Analysis system according to claim 1, wherein the electrodes are made from the same metal or different metals, for example titanium or platinum.

9. Analysis system according to claim 1, wherein the first detection substance (E-1) and/or the second detection substance (E-2) is selected from the group: calmagite, dimethylglyoxime, calcon carboxylic acid, xylenol orange, eriochrome black T, eriochrome blue black R, ethylenediamine tetraacetate (EDTA), oxalic acid, Kalignost, murexide, methylthymol blue, metal phthalein, pyrocatechol violet, 1-(2-Pyridylazo)-2-naphthol, 4-(2-Pyridylazo)resorcinol, iron(III)chloride, or mixtures thereof.

10. Analysis system according to claim 1, wherein the cations are selected from the group: iron(II) ions, iron(III) ions, copper (II) ions, Magnesium (II) ions, calcium (II) ions, manganese (II) ions, potassium(I) ions, ammonium cations (NH₄⁺), or mixtures thereof, and/or wherein the anions as selected from the group: nitrate ions (NO₃⁻), nitrite ions (NO₂⁻), phosphate ions (PO₄³⁻), or mixtures thereof.

11. Analysis system according to claim 1, wherein the first and/or second liquid measuring chamber medium comprises an electrolyte, preferably sodium sulfate.

12. Analysis system according to claim 1, wherein the anion-selective membrane and/or the cation-selective membrane are each made from a membrane material that comprises:
a first polymer material, preferably modified polystyrene (PS), with functional groups arranged on a polymer material surface, wherein the functional groups arranged on the polymer material surface functional groups lend an anion selectivity to the anion-selective membrane and/or a cation selectivity to the cation-selective membrane,
wherein the functional groups arranged on the polymer material surface for lending anion selectivity to the anion-selective membrane are preferably quaternary amines, and/or wherein the functional groups arranged on the polymer material surface for lending cation selectivity to the cation selective membrane are preferably carboxylic acid or sulfonic acid groups,
a second polymer material for mechanical strengthening of the anion-selective membrane and/or the cation-selective membrane, preferably polyvinyl chloride (PVC), polypropylene (PP) or polyethylene terephthalate (PET).

13. Analysis process for qualitative and/or quantitative determination of ions in an ion-containing liquid medium, comprising the following steps:
S2: Photometric measurement of anions (A) and/or anion detection substance associates (A-E-1) present in the first measuring chamber with the measuring arrangement, and/or photometric measurement of cations (K) and/or cation detection substance associates (K-E-2) present in the second measuring chamber with the measuring arrangement,
S3: qualitative and/or quantitative determination of the anions (A) and/or cations (K) on the basis of the photometric measurement in step S2.

14. Analysis process according to claim 13, wherein the following step is performed prior to step S2:
SO-a: application of a voltage between the first electrode and the second electrode, whereby the first electrode is polarized positively and the second electrode is polarized negatively, and whereby anions (A) are transported from the sample chamber though the anion-selective membrane towards the first electrode arranged in the first measuring chamber, and cations (K) are transported from the sample chamber through the cation-selective membrane towards the second electrode arranged in the measuring cell.

15. Analysis process according to claim 13, wherein the following step is performed prior to step S2:
S0-b: adjustment of a pH of the first liquid measuring chamber medium contained in the first measuring chamber, and/or of the second liquid measuring chamber medium contained in the second measuring chamber with an analysis system according to claim 3.

16. Analysis process according to claim 14, wherein the following step is performed after step S3 or prior to step S0-a:
S4: application of a reverse voltage compared to the voltage applied in step S0-a between the first electrode and the second electrode, whereby the first electrode is polarized negatively and the second electrode is polarized positively,
whereby the anion detection substance associates (A-E-1) formed in the first measuring chamber in optional step S1 are reverted to anions (A) and the first detection substance (E-1), and anions (A) are transported from the first measuring chamber through the anion-selective membrane back into the sample chamber, whereby the first detection substance (E-1) is regenerated, and/or
whereby the cation detection substance associates (K-E-2) formed in the second measuring chamber in optional step S1 are reverted to cations (K) and the second detection substance (E-2), and cations (K) are transported from the second measuring chamber through the cation-selective membrane back into the sample chamber, whereby the second detection substance (E-2) is regenerated.

17. Analysis system according to claim 1, wherein the analysis system as a compact probe with integrated pump unit is arranged directly in a sample reservoir.

18. Analysis process according to claim 13, wherein the following step is performed prior to step S2:
S1: Formation of anion detection substance associates (A-E-1) from anions (A) present in the first measuring chamber and the first detection substance (E-1), and/or formation of cation detection substance associates (K-E-2) from cations (K) present in the second measuring chamber and the second detection substance (E-2).
